(19) United States
(12) Patent Application Publication
(10) Pub. No.: US 2016/0161509 A1
(43) Pub. Date: Jun. 9, 2016

(54) METHODS FOR PREDICTING EMBRYO VIABILITY

(71) Applicants: Paul H. Pevsner, Irving, TX (US); Frederick Naftolin, New York, NY (US); Jamie Grifo, New York, NY (US); Frederick Licciardi, Rivervale, NJ (US)

(72) Inventors: Paul H. Pevsner, Irving, TX (US); Frederick Naftolin, New York, NY (US); Jamie Grifo, New York, NY (US); Frederick Licciardi, Rivervale, NJ (US)

(21) Appl. No.: 15/042,439

(22) Filed: Feb. 12, 2016

Related U.S. Application Data
(63) Continuation of application No. 12/231,894, filed on Sep. 5, 2008, now abandoned.
(60) Provisional application No. 60/967,960, filed on Sep. 7, 2007.

(51) Int. Cl. G01N 33/76; G01N 33/50; G01N 33/68

(52) U.S. Cl. CPC G01N 33/76; G01N 33/689; G01N 33/5091; G01N 2333/59; G01N 2560/00

(57) ABSTRACT

The present invention provides methods for predicting the viability of an embryo or for predicting the likelihood of a negative outcome during pregnancy by identifying the presence or absence of or determining the amount of one or more pregnancy associated markers such as molecular isoforms of hCG in a sample. In many instances, the invention is applicable to embryos generated by in vitro fertilization techniques, for instance, to embryos developing in a growth media. The present invention further provides methods for determining the amount of a pregnancy associated markers such as molecular isoforms of hCG (hCG) in a sample. The present invention also provides a diagnostic kit for predicting the viability of an embryo or for predicting the likelihood of a negative outcome during pregnancy by identifying the presence of or determining the amount of one or more pregnancy associated markers such as molecular isoforms of hCG in a sample.

### FIELD OF THE INVENTION

This invention relates to methods of determining whether a fetus is likely to become viable and whether a pregnancy is likely to have a positive or negative outcome.

### BACKGROUND OF THE INVENTION

The first evidence of in vitro fertilization (IVF) dates back to the 1890's when embryos were transferred between rabbits in England. In the late 1950s rabbit oocytes were fertilized in vitro and subsequently transferred back to female rabbits. Based on these animal techniques, the procedures were applied to humans, and eventually the first “test-tube baby” was born in England on Jul. 25, 1978 (Steptoe et al., Lancet 1978; 2: 366). Subsequently, physicians around the world began performing IVF. The first successful IVF procedure in the United States was performed in 1981. Since then, IVF procedures have developed and success rates have increased dramatically. It is estimated that in the US, 1% of births are conceived after IVF.

According to statistics from a 1995 Center for Disease Control (CDC) survey, 6.1 million women aged 15-44 years in the US have an impaired ability to conceive and during that year, 9.3 million women sought fertility services. The Society of Assisted Reproductive Technology (SART) 2005 annual report indicates that 123,200 cycles of IVF were performed in women under the age of 43 in the US. This number includes the majority of fertility clinics in the country; however, a minority do not report to SART and therefore this number underestimates the total IVF cycles in the US for 2005.

In an IVF procedure, patients are started on ovarian stimulation protocols per their individual physician based on infertility etiology, physical exam, hormonal assays, and prior history. Specifically, patients are placed on down-regulated GnRH agonist (leuprolide) protocols, micro-dose leuprolide protocols, or GnRH-antagonist protocols with gonadotropins starting on day 2 or 3 of the menstrual cycle. The goal of ovarian hyperstimulation is to suppress a woman's endogenous ovulation and hyperstimulate the ovaries to make multiple oocytes without placing the woman at risk. Patients are monitored frequently with serum estradiol levels and transvaginal ultrasonography of the ovaries to assess follicular development. Gonadotropin doses are adjusted during the cycle to achieve adequate numbers of mature oocytes at retrieval while minimizing the risk of ovarian hyperstimulation syndrome. When lead follicles reach a mean diameter of 17-18 mm, human Chorionic Gonadotropin (hcG) 10,000 Iu is given and approximately 36 hours later, oocytes are collected by ultrasound guided, transvaginal aspiration.

Oocytes are immediately placed in Human Tubal Fluid media (HTF, Irvine Scientific, Irvine Calif.) supplemented with 6% Plasmanate (Plasma Protein Fraction (Human) 5%, USP, Bayer Co., Elkhart, Ind.) overlaid with Sage mineral oil (Cooper Surgical, Trumbull, Conn.). The partner's sperm is collected and washed on the day of retrieval. Oocytes are fertilized by standard insemination or intracytoplasmic sperm injection (ICSI) when indicated 4-6 hours post retrieval. Oocytes inseminated by ICSI are quickly placed in Quinn's Advantage^{R} Cleavage Media (Q1) (Cooper Surgical, Trumbull, Conn.). After fertilization, oocytes are incubated overnight. On the day following insemination, day 1 post retrieval, oocytes are assessed for fertilization. When 2 pronuclei (2pn) are observed, fertilization is confirmed. Embryos that are created after standard insemination are transferred to Q1 media. Embryos are incubated and not reassessed until day 3 post retrieval. At this time, if only several viable embryos are available, embryo transfer is scheduled on this day. However, when rapidly developing good quality embryos exist in excess to the number that would be safely transferred (generally>4 embryos), embryos are transferred to Quinn's Advantage^{R} Blastocyst (Q2) media and cultured to day 5. On day 5 post retrieval, embryos are assessed and the most morphologically advanced embryos are selected for transfer to the uterus. Excess embryos are transferred into a fresh dish of Q2 and incubated overnight. On day 6, embryos that meet strict morphologic criteria are selected for cryopreservation.

Patients undergo embryo transfer using a soft tipped catheter. In most cases ultrasound guidance is used to deposit the embryos approximately 1 cm from the uterine fundus. The decision on the number of embryos to transfer is based on embryo morphology and patient characteristics (age, history). The final decision on the number to transfer is made after a discussion between physician and patient. Patients are instructed to take intramuscular progesterone injections until day 28 (day 14 established as day of oocyte retrieval) when the initial pregnancy test is obtained. In general, progesterone is continued until a fetal heartbeat is observed on ultrasound. Patients with a positive pregnancy test are followed for the development of a viable intrauterine pregnancy and for ectopic pregnancies.

Human preimplantation embryos cultured in vitro are characterized by variable morphology and developmental potential. Following in vitro fertilization (IVF), an average of 25% of transferred embryos implant and, an average of 20% of women become pregnant. The established criteria for human embryo viability are histological patterns of 3 and 5 day embryos identified microscopically by experienced reproductive physicians and embryologists. Implant decisions made with these criteria can achieve successful implantation in half of the implants and a top rate of 50% live births. In vitro studies show that during the first six days of preimplantation development approximately 50% of human embryos arrest. Varying degrees of cytoplasmic fragmentation occurs in approximately 75% of human embryos. This is associated with reduced blastocyst formation and implantation. The causes, mechanism of fragmentation and precise reasons for early embryonic loss remain unknown. Cell death has been observed during preimplantation embryogenesis both in vivo and in vitro in a range of mammalian species. Cell death is prevalent in human blastocysts with approximately 75% of embryos having one or more dead cells on day 6. Cells and nuclei with the morphological features of apoptosis which include intracellular fragmentation, chromatin condensation, DNA fragmentation and phagocytosis have been identified in human preimplantation embryos. Apoptosis may be involved in early embryonic arrest, and cytoplasmic fragments are equivalent to apoptotic bodies, i.e., the end product of apoptosis. Suboptimal culture conditions may be implicated in preimplantation arrest and cell death. In particular, there is increasing evidence that growth factors play an important role in preimplantation development. For example, the preimplantation development of mouse embryos cultured in vitro is retarded when compared to that in vivo. Moreover, reduced incubation volume or cultures of embryos in groups improve preimplantation development. Furthermore, a range of polypeptide growth factor ligands and their receptors have been found to be expressed and produced in the reproductive tract or preimplantation embryo, including epidermal growth factor (EGF), transforming growth factor alpha (TGF-a), insulin receptor insulin-like growth factor I (IGF-I) and its receptor (IGF-IR). Generally, growth factors have been shown to promote blastocyst formation and development and increase cell number. Taken together, these findings suggest the presence of regulatory autocrine and paracrine pathways acting in vivo that may be ‘diluted’ or not present in the in vitro environment. (Hardy, K. et al., PNAS 2001, 98(4):1655-1660; Spanos et al., Biology of Reproduction 2000, 63:1413-1420).

The scope of IVF and other forms of assisted reproduction technologies (ART) in the United States is over 100 000 IVF cycles, and ART babies now account for approximately 0.6% of all births in this country. A small but important percentage of these children suffer from a variety of significant morbid congenital problems. Children conceived by ART have twice the rate of major birth defects as compared with babies conceived naturally. This risk persists even after adjusting for increased maternal age and increased incidence of multiple gestations with ART. A wide range of birth defects has been noted, including chromosomal abnormalities, musculoskeletal and cardiovascular defects, and low birthweight. (Li, Tao et al., Molecular Human Reproduction, 2005, 11(9):631-640).

The ability to identify the most viable chromosomal normal embryos for transfer is of fundamental importance to assisted reproduction techniques (ARTs). An early predictive assessment made in a non-invasive manner would allow ART clinics to identify the best embryo that would result in a live birth.

Human chorionic gonadotropin (hCG) is a glycoprotein with 8 oligosaccharide side chains. Sugar residues account for approximately 30% of the molecular weight of hCG, and variation in oligosaccharide branching is a key factor in the hCG structure (Elliott et al., 1997, Endocrine 7:15-32). hCG exists in maternal urine in various forms including hyperglycosylated hCG, also called invasive trophoblastic antigen (ITA). While hCG is produced by differentiated syncytiotrophoblast cells, ITA is produced solely by invasive cytotrophoblast cells (Kovalevskaya et al., 2002, Mol Cell Endocrinol 194:147-55; Lei et al., 1999, Troph Res 13:147-59). ITA, which is the predominant form of hCG produced in invasive trophoblast disease and early pregnancy at the time of and following implantation, contains additional antennae on the oligosaccharide side chains (O'Connor et al., 1998, Prenat Diagn 18:1232-40). Each of the side chain antennae normally ends with a sialic acid residue. The extent of sialic acid content on the ITA molecule is dependent on abundance of sialic acid in the body. Phosphoenolpyruvate, which is a major intermediate in sugar metabolism, is a substrate in the production of sialic acid (Elliott et al., 1997, Endocrine 7:15-32). Thus, depending on cellular growth and metabolism, ITA can vary greatly in sialic acid content, or charge; it is generally more deficient in this acidic sugar than is hCG (Elliott et al., 1997, Endocrine 7:15-32). Normally-glycosylated hCG contains between 11 and 15 sialic acid residues, whereas ITA can contain between 8 and 19 sialic acid residues (Elliott et al., 1997, Endocrine 7:15-32). Sialic acid contributes to the biological activity of the molecule by protecting terminal galactose residues from liver galactose receptors, by increasing the circulating half-life of the molecule, and by stabilizing the hCG .alpha.-.beta. dimer (Brand et al., 1980, Acta Endocrinologica 95:75-83; Rosa et al., 1984, J Clin Endocrinol Metab 59:1215-9; Van Hall et al., 1970, Endocrinology 89:11-15).

The various isoforms of hCG have received increasing attention during recent years for their potential diagnostic value in problem pregnancies such as Down syndrome, preeclampsia, trophoblastic diseases and early pregnancy loss. Nicked hCG at high concentrations has been associated with trophoblastic disease and other abnormal states of hCG production. Nicked hCG has been measured by a variety of qualitative techniques such as immunoblotting, as well as by direct isolation and sequence analysis of such hCG isoforms from urine.

At least two factors affect increased potency of hCG. First, it is known that a larger Stoke's radius will decrease clearance through the kidney glomerulus which generally clears proteins above an effective size of 70,000 very slowly. The effective size of urinary-isolated hCG is just at this borderline reduced clearance size. Generally, extra sugar content makes the hydrated radius of glycoproteins larger. It has been shown that by adding the hCG beta COOH-terminal peptide to hFSH or hLH, their circulating life-times greatly increased. This addition was thought mostly due to the carbohydrate content of that peptide rather than simply the extra polypeptide size. Second, increased negative charge of a protein will prolong its circulating time because of decreased renal clearance. This increased negative charge can be due to extra sialic acid or other negative groups, including sulfate such as is present on hLH and on the pituitary form of hCG. Changes which affect signal transduction at the receptor may also affect biopotency of hCG. It is known that deglycosylated hCG has much reduced receptor potency. Carbohydrate reduced forms of hCG also have reduced signal transduction.

### SUMMARY OF THE INVENTION

The present invention provides a method of predicting whether a fetus is likely to become viable by determining whether one or more pregnancy associated markers is present in a sample. The pregnancy associated marker may be a molecular isoform of hCG. One, two, three, four, five, six, seven or eight or more of the pregnancy associated markers such as molecular isoforms of hCG may be present in the sample and may be identified. In some embodiments it is the absence or relative absence of one or more pregnancy associated markers that is predictive of the likelihood of a viable fetus.

The sample used for determining the presence of one or more molecular isoform of hCG may be an in vitro growth media, amniotic fluid, plasma, serum, urine or blood. The one or more pregnancy associated markers such as molecular isoforms of hCG may be identified by many methods well known to those of skill in the art including Matrix assisted laser desorption ionization (MALDI) mass spectrometry. The pregnancy associated markers such as molecular isoforms of hCG may also be identified by contacting the sample with an antibody which specifically binds to the molecular isoform of hCG under conditions permitting formation of a complex between the antibody and the molecular isoform of hCG, and optionally measuring the amount of complexes formed, thereby determining the amount of the molecular isoform of hCG in the sample.

The methods may optionally include quantifying one or more of the pregnancy associated markers such as molecular isoforms of hCG. The methods may further include comparing the amount of molecular isoform of hCG in the sample determined to be present in the sample with either (i) the amount determined for temporally matched, normal samples or (ii) the amount determined for samples obtained from non-pregnant subject(s) when the sample is any other than an in vitro growth media. The relative absence of one or more of the pregnancy associated markers such as molecular isoforms of hCG in the sample indicates that the embryo is relatively non viable and the relative abundance of one or more of the pregnancy associated markers such as in the sample indicates that the embryo is relatively viable in some embodiments. Conversely, in other embodiments, the relative absence of one or more of the pregnancy associated markers such as molecular isoforms of hCG in the sample indicates that the embryo is relatively viable and the relative abundance of one or more of the pregnancy associated markers in the sample indicates that the embryo is relatively non-viable.

The methods of the present invention are applicable to determining viability of an embryo at many stages in its development, whether in vitro or in vivo. In most instances, the embryo is a mammal, especially a human, and in most instances the embryo is in a relatively early developmental stage within the first trimester of pregnancy. The embryo may be, for instance, three months, two months, one month, two weeks, one week, five days, three days or one day post fertilization. In some embodiments, the embryo is a product of in vitro fertilization. In some of these embodiments, the embryo has yet to be implanted and is growing in an in vitro growth media.

The present invention further provides a method of predicting pregnancy outcome in a subject. In some embodiments, it is a method of predicting the likelihood of a positive pregnancy outcome in a female subject comprising determining the presence of one or more pregnancy associated markers such as molecular isoforms of hCG in a sample. In other embodiments, it is a method of predicting the likelihood of a negative pregnancy outcome in a female subject comprising determining the absence or the relative absence as compared to normal samples or normal subjects of one or more pregnancy associated markers such as molecular isoforms of hCG in a sample. One, two, three, four, five, six, seven or eight or more of the pregnancy associated markers such as molecular isoforms of hCG may be present in the sample and may be identified. One, two, three, four, five, six, seven or eight or more of the pregnancy associated markers such as molecular isoforms of hCG may be present in the sample and may be elevated in comparison to samples obtained from normal pregnant subjects.

The sample used for determining the presence of one or more molecular isoform of hCG may be an in vitro growth media, amniotic fluid, plasma, serum, urine or blood. The one or more pregnancy associated markers such as molecular isoforms of hCG may be identified by many methods well known to those of skill in the art including Matrix assisted laser desorption ionization (MALDI) mass spectrometry. The pregnancy associated markers such as molecular isoforms of hCG may also be identified by contacting the sample with an antibody which specifically binds to the molecular isoform of hCG under conditions permitting formation of a complex between the antibody and the molecular isoform of hCG, and optionally measuring the amount of complexes formed, thereby determining the amount of the molecular isoform of hCG in the sample.

The methods may optionally include quantifying one or more of the pregnancy associated markers such as molecular isoforms of hCG. The methods may further include comparing the amount of pregnancy associated markers such as molecular isoforms of hCG in the sample determined to be present in the sample with either (i) the amount determined for temporally matched, normal samples or (ii) the amount determined for samples obtained from non-pregnant subject(s) when the sample is any other than an in vitro growth media. The relative absence of one or more of the molecular isoforms of hCG in the sample indicates that the likelihood of a negative outcome is greater than the normal, and the relative abundance of one or more of the molecular isoforms of hCG in the sample indicates that the likelihood of a positive outcome is greater than the normal.

The invention provides methods for determining whether a fetus is likely to be viable and methods for determining the relative likelihood of a positive or negative outcome for a pregnant woman comprising the steps of: (a) obtaining a biological sample from a pregnant woman or from a growth media in instances where the fetus is not in vivo; (b) measuring an amount of one or more pregnancy associated hCG isoforms present in the biological sample; and (c) comparing the amount of pregnancy associated hCG isoforms with a predetermined value, whereby the amount of pregnancy associated hCG isoforms relative to the predetermined value indicates whether a fetus is likely to be viable or the relative likelihood of a positive or negative outcome for a pregnant woman.

In one embodiment, the method further comprises measuring an amount of one or more pregnancy associated markers such as hCG isoforms in the biological test sample; and calculating the ratio of the amount of the one or more pregnancy associated markers such as hCG isoforms to the amount of other forms of pregnancy associated markers such as hCG isoforms, whereby the ratio indicates whether a fetus is likely to be viable or the relative likelihood of a positive or negative outcome for a pregnant woman.

Further, the present invention provides a diagnostic kit for assessing viability of an embryo by determining the presence or absence or by quantifying the amount of one or more pregnancy associated markers such as molecular isoforms of hCG in a biological sample.

### BRIEF DESCRIPTION OF THE FIGURES

FIG. 1 depicts the results of seven MALDI ms spectra obtained from an embryo growth media fluid Q2, a combination of Quinn's Media and 10% human pooled plasma as Plasmanate© in which embryos are grown. The embryos are identified as follows: line 1 High potential embryo>live baby; line 2 High potential embryo>live baby; line 3 Low potential embryo>bad embryo now growth, discarded; line 4, High potential embryo>live baby; line 5, Low potential embryo>bad embryo now growth, discarded; line 6, Low potential embryo>bad embryo now growth, discarded; and line 7, Q2 growth media blank. Only the high potential embryos (actively growing and developing) were able to utilize the proteins identified in the low potential embryos and the blank: molecular weight 9149-9157. The ability to utilize/metabolize this protein is evidence of a high potential embryo.

### DETAILED DESCRIPTION OF THE INVENTION

As used herein, the following terms mean as follows:

As used herein, “pregnancy associated marker” means any molecule, such as a protein, peptide or fragment thereof whose presence, absence or amount in absolute quantity or in quantity relative to other molecules may be used as evidence of whether an embryo is likely to be viable or whether a pregnancy is likely to have a positive or a negative outcome. Stated differently, a pregnancy associated marker is any molecule that may be used as a statistically significant predictor of viability or pregnancy outcome.

As used herein, “hCG” is an abbreviation for human chorionic gonadotropin, a glycoprotein hormone secreted in relatively large quantities by the trophoblast cells of the placenta. hCG, also referred to herein as “normal hCG,” is composed of two dissimilar subunits, α (92 amino acids and two N-linked oligosaccharides) and β (145 amino acids and two N-linked and four O-linked oligosaccharides), joined noncovalently, and is detected in the serum and urine of pregnant women and in those with trophoblast disease (such as hydatidiform mole or choriocarcimoma). Free α- and free β-subunits are also detected in serum and urine samples.

As used herein, a “predetermined value” is a standardized value based on a control. For example, a predetermined value can be based on an amount of pregnancy associated hCG isoforms that are present in a biological sample obtained from a pregnant woman who carries a normal fetus. In this embodiment of the invention, a fetus may be determined likely to be non-viable or a pregnancy deemed likely to have a negative outcome if the amount of one or more pregnancy associated hCG isoforms is lower than the predetermined value. Similarly, a fetus may be determined likely to be viable or a pregnancy deemed likely to have a positive outcome if the amount of one or more pregnancy associated hCG isoforms is higher than the predetermined value.

As used herein, an “hCG isoform” is a hyperglycosylated variant of hCG, including, but not limited to, for instance, ITA and ITA-2.

The term “amount” is used within the context of the analytical method used to measure the different pregnancy associated markers such as molecular isoforms of hCG and may reflect a number, a concentration, etc., depending upon the analytical method chosen to measure the pregnancy associated markers such as molecular isoforms of hCG.

The term “biological sample,” as used herein, generally refers to urine, saliva, serum, plasma, tears, or amniotic fluid as well as in vitro growth media.

The term “detecting” as used herein refers to identifying the presence of, identifying the presence of in relative amounts relative to another molecule or pregnancy associated markers such as molecular isoforms of hCG or relative to a predetermined value, or quantifying in absolute amounts.

The term “positive outcome” in relation to pregnancy means delivery of a baby substantially free of genetic abnormalities or diseases.

The term “negative outcome” in relation to pregnancy means failure to deliver a baby at all, abortion or delivery of a baby substantially impacted or afflicted by genetic abnormality or disease.

The present invention further provides human chorionic gonadotropin hormone (hCG) isoforms associated with viable and non-viable embryos as well as heavy chain factor VII inhibitor associated with live embryos. The present invention identifies proteins associated with viable embryos and successful implantation from 3 and 5-day embryo culture media with Matrix assisted laser desorption ionization (MALDI) mass spectrometry.

The established criteria for determining human embryo viability are histological patterns identified microscopically by experienced reproductive physicians and embryologists. Implant decisions made with these criteria can achieve successful implantation in half of the implants.

The present invention utilizes, in some embodiments, MALDI mass spectrometry to identify proteins associated with embryo viability from growth media fluid. Reduced, alkylated, and trypsinized aliquots of control and 19 embryo culture media obtained from three and five-day embryos were studied with MALDI. The following isoforms of hCG were identified and associated with single implant viable embryos.
gi|119606107 hCG2045454 [Homo sapiens]
gi|119571712 hCG2038841 [Homo sapiens]
gi|119602728 hCG1765609 [Homo sapiens]
gi|119615374 hCG2045605 [Homo sapiens]

In some embodiments the methods provide for comparing the amount of pregnancy associated molecular isoform of hCG in a sample with either (i) the amount determined for temporally matched, normal pregnant subject(s) or (ii) the amount determined for non-pregnant subject(s), wherein amounts of the pregnancy associated molecular isoform of hCG in the sample similar to amounts of pregnancy associated molecular isoform of hCG in temporally matched pregnant samples indicates a positive outcome, amounts of early pregnancy associated molecular isoform of hCG in the sample similar to amounts of pregnancy associated molecular isoform of hCG in the non-pregnant samples indicates a negative outcome of pregnancy for the subject.

According to an embodiment of this invention, the sample may be a urinary sample, a sample of amniotic fluid or a blood sample. In one embodiment of this invention, the sample is an aggregate sample taken from at least two consecutive days. In an embodiment of this invention, the sample is a spot urine sample, a first morning void urine sample, or an aggregate sample of the first morning void urine samples for at least two consecutive days. In one embodiment of this invention, the antibody is labeled with a detectable marker. In an embodiment of this invention, the detectable marker is a radioactive isotope, enzyme, dye, magnetic bead, or biotin.

In addition, the present invention provides a method for determining the amount of pregnancy associated molecular isoforms of in a sample comprising: (a) contacting the sample with an antibody which specifically bind to a pregnancy associated molecular isoform of hCG under conditions permitting formation of a complex between the antibody and the pregnancy associated molecular isoform of hCG; and (b) determining the amount of complexes formed thereby determining the amount of pregnancy associated molecular isoform of hCG in the sample.

Unexpected isoforms of hCG are produced during normal pregnancy. These isoforms may have enhanced potency for signal transduction. These isoforms can help predict pregnancy outcome where one cause of negative pregnancy result is failure to produce the isoform of hCG of higher potency produced by successful pregnancies. Identification of the nature of the hCG isoform required might provide the proper reagent needed to sustain pregnancy.

Forms of hCG, which have greatly reduced bioactivity may contribute to a lack of embryo viability or negative pregnancy results, due at least in part to diminished biopotency.

This invention is illustrated in the Experimental Details section which follows. These sections are set forth to aid in an understanding of the invention but are not intended to, and should not be construed to, limit in any way the invention as set forth in the claims which follow thereafter.

hCG, hCG isoforms and pregnancy associated hCG isoforms can be directly measured, for example, using anti-hCG, hCG isoforms and pregnancy associated hCG isoforms antibodies in an immunoassay, such as a Western blot or ELISA. Anti-hCG, hCG isoform and pregnancy associated hCG isoform antibodies can be generated as described herein. hCG, hCG isoforms and pregnancy associated hCG isoforms can be indirectly measured, for example, using an hCG capture antibody that binds hCG and various hCG isoforms, followed by either carbohydrate analysis, which will distinguish hCG isoforms from hCG by variations in, for instance, sialic acid content, or by removing normal hCG and hCG isoforms using anti-hCG antibodies and anti-hCG antibodies and quantifying remaining protein, which may consist of hCG isoform protein. Alternatively, hCG, hCG isoforms and pregnancy associated hCG isoforms can be indirectly measured, for example, using a capture antibody that recognizes hCG isoforms, such as, for instance, hyperglycosylated hCG. An example of a monoclonal antibody that recognizes the hCG isoform ITA is B-152, described in U.S. Pat. No. 6,339,143, relevant portions of which are incorporated by reference herein. ITA-2 is discussed in Sutton and Cole, Prenatal Diagnosis, 24:194-197 (2004) and Down Syndrome News, 10:32 (2003), relevant portions of which are incorporated by reference herein.

The methods of the invention can be used alone or in combination with any known test for assessing viability of a fetus or for determining prognosis for a pregnancy, including, but not limited to, a triple screen test (combination of maternal age with serum measurements of hCG, α-fetoprotein (AFP), and unconjugated estriol), unconjugated and/or conjugated estriol measurements, hCG assays, β-core fragment analyses, free β-subunit or free α-subunit analyses, PAPP-A or CA125 analyses, α-fetoprotein analyses, inhibin assays, observations of fetal cells in serum, and ultrasound. The methods of the invention can be used to screen a biological sample collected during the first, second, or third trimester of pregnancy or before in vivo implant. In addition, the methods of the invention can be combined with any known diagnostic test during the first, second, or third trimester of pregnancy.

The amount of pregnancy associated hCG isoforms in a biological sample can be determined using any method known in the art, including, but not limited to, immunoassays using antibodies specific for various hCG charge isoforms, isoelectric focusing, carbohydrate analysis, matrix assisted laser desorption/ionization (MALDI), or some combination thereof. For example, highly acidic and less acidic hCG isoforms can be distinguished in a biological sample by first preparing the sample for isoelectric focusing by diluting the sample with ampholytes and separating the proteins in the sample by charge using, for example, a Rotofor® Cell (Bio-Rad, Hercules, Calif.). Then, fractions of proteins can be collected at particular isoelectric point (pI) ranges, and hCG isoforms in each fraction of various pI ranges can be quantitatively determined.

Any assay that functions to qualitatively or quantitatively determine variations in sample concentrations of hCG from normal levels, and/or detects hCG isoforms in the sample's gonadotropin population can be employed in the practice of the invention. A direct assay, such as an immunoassay using antibodies that recognize hCG or specific hCG isoforms, is preferred, but other exemplary assays can involve lectins that assay for carbohydrate moieties or any other fingerprinting technique including qualitative or quantitative carbohydrate composition analysis, chromatography, chemical or electrophoresis or isoelectric focusing tests, among others, or any other methods that detect glycosylation variants of hCG, and/or antibodies to hyperglycosylated or carbohydrate-variant hCG. Such assays are described in the art.

Immunoassays that can be used to detect hCG or hCG isoforms include, but are not limited to, assays employing specific antibodies to hCG or isoforms thereof, and assays employing nonspecifically defined antibodies. Antibodies to hCG and hCG isoforms, such as ITA and ITA-2, can be generated by standard means as described, for example, in “Antibodies: A Laboratory Manual” by Harlow and Lane (Cold Spring Harbor Press, 1988), which is hereby incorporated by reference.

For example, a monoclonal anti-hCG isoform antibody can be generated by immunizing a mouse with recombinant hCG isoform, purified hCG isoform, hCG isoform pre-treated with neuramimidase, or a cell expressing recombinant hCG isoform, purified hCG isoform, or hCG isoform pre-treated with neuramimidase. Once an immune response is detected, e.g., antibodies specific for the hCG isoform are detected in the mouse serum, the mouse spleen is harvested and splenocytes are isolated. The splenocytes are then fused by well-known techniques to any suitable myeloma cells, for example, cells from cell line SP20 available from the American Type Culture Collection (ATCC). Hybridomas are selected and cloned by limited dilution. The hybridoma clones are then assayed by methods known in the art for cells that secrete antibodies capable of binding the hCG isoform. Ascites fluid, which generally contains high levels of antibodies, can be generated by immunizing mice with positive hybridoma clones.

Any type of fusion phage, monoclonal, or polyclonal antibodies can be used in immunoassays of the invention, so long as the antibodies can be used in a reproducible fashion as markers for various hCG isoforms or as measures of the different levels of hCG isoforms observed in normal and variant populations.

In one embodiment, an amount of hCG or a hCG isoform can be measured using a capture antibody followed by a labeled secondary antibody using a strategy as described, for example, in U.S. Pat. No. 6,429,018, hereby incorporated by reference. U.S. Pat. No. 6,429,018 teaches the B152 antibody that recognizes nicked hyperglycosylated hCG but does not detect normal hCG. A labeled secondary antibody useful in a method of the invention (for example, as taught by U.S. Pat. No. 6,339,143 can be, for example, an anti-hCG antibody, β-core fragment, α-subunit, and/or hCG isoform β-subunit, providing assay with polypeptide specificity. The label on the secondary antibody can comprise any chemical, radioactive, lanthanide, colored dye, or genetic tag used in enzyme-linked immunosorbent assays (ELISAs), Western blots, and other sensitive and specific immunoassays and immunoradiometric assays using known methodology. These include conjugating the antibody with horseradish peroxidase or alkaline phosphatase that are easily measurable, typically using colorimetric, fluorometric or luminescent substrates. Genetic labels include firefly luciferase, employed because luciferase produces a bioluminescent molecule when incubated with its substrate, luciferin.

In other embodiments, hCG peptide-specific antibody can be used as a capture antibody, and an antibody specific to hyperglycosylated or carbohydrate-variant hCG and/or an abnormal carbohydrate portion thereof can be used as the secondary labeled antibody in an immunoassay such as those described above. Competitive immunoassays employing antibodies to specific hCG isoforms can also be employed to competitively detect hCG isoforms. Alternate embodiments using concanavalin A or other carbohydrate-specific lectin can be used in place of the capture antibody or labeled antibody. Alternatively, prior to an immunoassay, a lectin or chromatographic method can be used to extract carbohydrate-variant hCG isoforms from a biological sample or from a fraction that was separated or pooled according to pI range. These methods are all well known in the art.

Carbohydrate analyses include qualitative observations of differences in physical properties between normal and Down's syndrome hCG populations, carbohydrate identification using plant lectins specific to the variant carbohydrate portion of hCG isoforms obtained by standard lectin screening methods, or any other fingerprinting technique including qualitative or quantitative carbohydrate composition analyses such as, for example, those described in U.S. Pat. No. 6,429,018, incorporated by reference herein.

In certain embodiments, hCG or hCG isoforms can be purified from biological samples prior to separating the hCG isoforms by charge distribution. Any method for purifying hCG can be used. For example, antibodies specific for hCG or specific hCG isoforms can be used to isolate hCG or hCG isoforms from a biological sample. A purified hCG protein fraction can be subjected to isoelectric focusing or any other method described herein to determine the amount of isoforms present in the samples having various pI values or ranges. Purified hCG and hCG isoforms from control or test samples can be stored under appropriate conditions, such as those described, for example, in Cole et al., 1999, Clinical Chem. 45:2109-2119, which is hereby incorporated by reference.

### EXAMPLE 1

Materials and Methods

IRB approved protocols were obtained to study murine and human in vitro fertilization growth media with MALDI mass spectrometry. An average of 15 μL of growth media per embryo was lyophilized to 1 μL. Cibicron on agarose beads 0.5 μL was added to the growth media to remove albumin, the mixture centrifuged, and the supernatant removed. Dithiothreital (DTT) 1 μL was added to the supernatant and incubated for one hour at 37° C. Iodoacetamide 2.5 μL was added to this mixture and incubated for one hour in the dark. Trypsin 1 mcg per microliter, was added to this solution and incubated for one hour at 37° C. The mixture was lyophilized to 1 μL. The lyophilized mixture 1 μL was placed on any MALDI target plate and covered with 1 μL of alpha cyano 4 hydroxy cinnamic acid. MALDI mass spectrometry was performed in reflectron mode, and either peptide mass fingerprinting, or MALDI tandem mass spectrometry performed with database identification of proteins by a Mascot© search engine.

Results

Matrix assisted laser desorption ionization mass spectrometry (MALDI) of in-vitro fertilization embryo growth media was used to identify multiple proteins. In a study of consecutive mouse embryos, the following proteins were identified gi|26347033, unnamed protein product [Mus musculus], gi|74178131, unnamed protein product [Mus musculus], gi|94379793, PREDICTED: hypothetical protein LOC338354 isoform 17 [Mus musculus], gi|47564082, hypothetical protein LOC407812 [Mus musculus], gi|74201402, unnamed protein product [Mus musculus], gi|6753480, procollagen, type X, alpha 1 [Mus musculus]. In human embryos the following proteins were identified: gi|7531054, Protein BCE-1, gi|5730196, Kruppel-type zinc finger [Homo sapiens], gi|119629659, chromosome 13 open reading frame 8, isoform CRA_b [Homo sapiens].

gi|223976 haptoglobin Hp2, gi|113421639, PREDICTED: similar to nuclear DNA-binding protein [Homo sapiens], gi|112696788, immunoglobulin heavy chain variable region [Homo sapiens], gi|31981810, dodecenoyl-Coenzyme A delta isomerase (3,2 trans-enoyl-Coenzyme A isomerase) [Mus musculus], and multiple isoforms.

### EXAMPLE 2

Materials and Methods

Patients who provided IRB consent to use of their discarded fluids (serum, culture media) were enrolled in the study. Inclusion criteria consisted of donor egg recipient patients who provided consent. Women were referred to donor egg for a variety of indications including: premature ovarian failure, repeated failed autologous oocyte IVF cycles, and maternal age in excess of 44 years. Routinely, after routine medical, psychological, and genetic screening, couples are placed on the donor egg waiting list where they are subsequently matched with an anonymous oocyte donor (woman aged 21-32 years). In some cases, couples assign a directed donor (sibling, friend). Donors undergo ovarian hyperstimulation with exogenous gonadotropins similar to IVF patients. These patients are also closely monitored with serum estradiol levels and serial pelvic ultrasounds. Similar to IVF patients, human Chorionic Gonadotropin (hCG) 10,000 Iu is given when lead follicles are 17 mm and transvaginal oocyte retrieval is performed approximately 36 hours later. Male sperm is collected and washed on the day of retrieval and insemination or ICSI is performed as in standard IVF. The donor egg recipient is started on leuprolide to suppress endogenous folliculogenesis and ovulation before the donor is started on stimulation medications. Once the donor starts the gonadotropins, recipients are started on exogenous estradiol to stimulate the uterine lining. Recipients are monitored for response to the estradiol and they are started on intramuscular progesterone and oral antibiotic and steroids the day the donor receives the hcG trigger. Typically, recipients undergo embryo transfer on day 5 after retrieval. The procedures may be summarized as in Table 1.

TABLE 1
Summary:

| | |
|---|---|
| Day 2/3 | Start gonadotropins for average of 10 days |
| (of menstrual cycle): | |
| Day 14: | Oocyte retrieval, ICSI/insemination (day 0 for |
| | embryo) |
| Day 1: | Fertilization check, 2pn embryos transferred into |
| | Q1 media |
| Day 2: | Incubate |
| Day 3: | Initial assessment, possible embryo transfer |
| | versus continued culture in Q2 media |
| Day 4: | Incubate |
| Day 5: | Final assessment, transfer 1-3 embryos based on |
| | embryo morphology and patient characteristics. |
| Day 6: | Excess embryos assessed for cryopreservation. |
| | Top quality embryos cryopreserved. |

Results

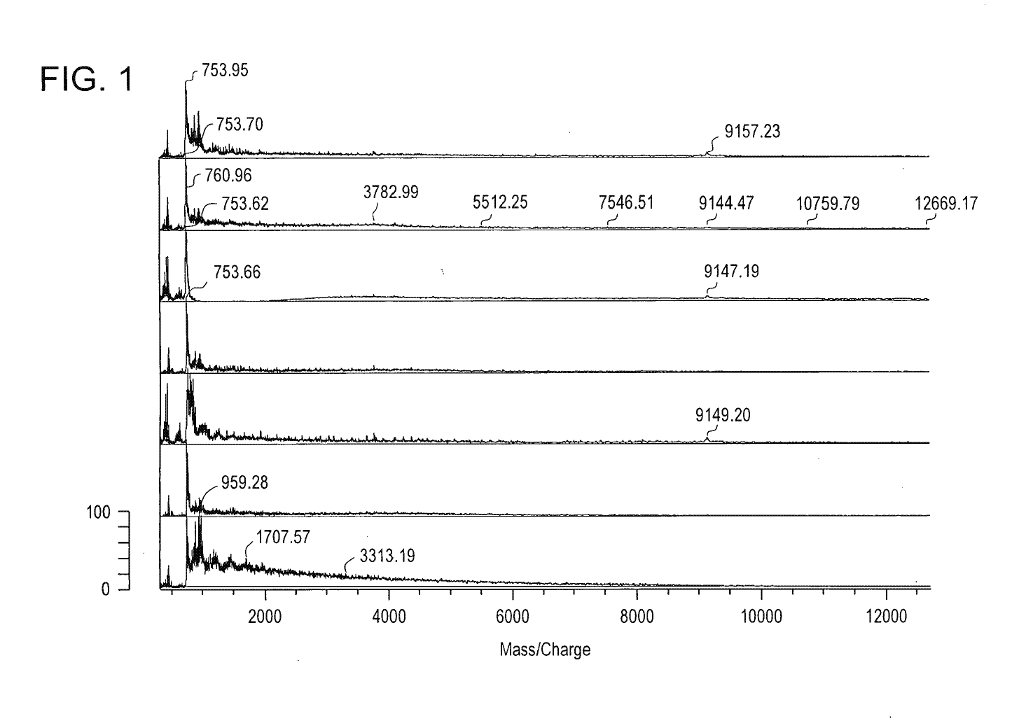

The seven MALDI ms spectra depicted in FIG. 1 are all obtained from the embryo growth media fluid Q2, a combination of Quinn's Media and 10% human pooled plasma as Plasmanate© in which all of these embryos were grown. They are identified as follows:
1. High potential embryo>live baby
2. High potential embryo>live baby
3. Low potential embryo>bad embryo now growth, discarded
4. High potential embryo>live baby
5. Low potential embryo>bad embryo now growth, discarded
6. Low potential embryo>bad embryo now growth, discarded
7. Q2 growth media blank.

Only the high potential embryos (actively growing and developing) were able to utilize the proteins identified in the low potential embryos and the blank: molecular weight 9149-9157. The protein is identified by Mascot search as gi|19629761 hCG2036844 isoform CRAb from Homo sapiens. The ability to utilize/metabolize this protein is evidence of a high potential embryo.

Discussion

For the initial decades following the first IVF baby in the US, physicians and researchers focused on improving the technique and ultimately improving success rates. In the most recent SART (2005) review, 37%, 29%, 20%, and 11% of IVF cycles in women under the age of 35, 35-37, 38-40, and 41-42, respectively, result in a live birth. These statistics vary from clinic to clinic. For example, at the NYU Fertility Center, 48%, 38%, 28%, and 12% of cycles in women <35 yrs, 35-37 yrs, 38-40 yrs, and 41-42 yrs, respectively, result in a live birth. Furthermore, 40% of these births in women <35 years at the NYU Fertility Center are twin deliveries. One of the greatest criticisms of assisted reproductive technologies (ART) is the high incidence of multiple and high order multiple gestations. In 2004, 139,494 infants were born from multiple gestation pregnancies in the United States. (Martin et al., Births: final data for 3004. National vital statistics reports, vol 55, no. 1. Hyattsville, Md.: National Center for Health Statistics, 2006) Since 1980, there has been an 93% increase in twins and 544% increase in triplet and higher order births. The rise of ART is largely responsible for these changes. It is well established that fetuses and mothers of multiple and high-order multiple pregnancies are faced with increased morbidity and mortality.

Grifo et al. recently described their clinic's progression to blastocyst transfer as a means to reduce the high-order multiple rate. (Grifo et al., Fertil Steril 2007, 88:294) Nonetheless, they still report a high rate of twin pregnancy. The ART community has addressed the need for more single embryo transfers (SET) but also recognizes the lowered pregnancy rates that may ensue. While many physicians may be tolerable of lower pregnancy rates, often patients are not given the high monetary and psychological costs of IVF. Efforts to identify embryos for SET have focused almost exclusively on morphologic and patient characteristics. Criniti et al report significantly lower twin pregnancies with comparable pregnancy rates in women <38 years with advanced blastocysts who underwent elective SET. (Criniti et al., Fertil Steril 2005, 84:1613) Nonetheless, the criteria identified to recommend SET has a narrow scope.

Preimplantation genetic screening (PGS) to identify euploid embryos was originally thought to be a promising technique to identify a single embryo with the greatest potential to result in a healthy pregnancy. However, the data has not been able to support improved IVF outcome with PGS and in fact, a recent prospective randomized study reported lower pregnancies rates in women undergoing PGS. (Mastenbroek et al., NEJM 2007, 357: 9) While this study was not perfect, it does not appear at this time that PGS will serve a successful tool for selecting embryos for SET.

The ability to identify additional markers or factors associated with embryo viability and success has been the greatest challenge towards promoting SET. The presence of secreted proteins in embryonic culture media is an area of research that appears promising. Particular proteins can be associated with successful pregnancy, and these proteins can be identified in culture media on day 3 allowing for transfer on day 5. This procedure is very advantageous because it does not require manipulation of the embryo as in PGS. Furthermore, it may be done quickly, allowing for timely transfer of embryos

We claim:

1. A method of predicting viability of an embryo comprising determining the presence of one or more pregnancy associated markers such as molecular isoforms of hCG in a sample.

2. A method according to claim 1 wherein the pregnancy associated marker is a molecular isoform of hCG.

3. A method according to claim 1 wherein the sample is selected from the group consisting of an in vitro growth media, amniotic fluid, plasma, serum, urine and blood.

4. A method according to claim 1 wherein determining the presence of one or more pregnancy associated markers in a sample is performed by Matrix assisted laser desorption ionization (MALDI) mass spectrometry.

5. A method according to claim 1 wherein determining the presence of one or more pregnancy associated markers in a sample is performed by contacting the sample with an antibody which specifically binds to a molecular isoform of hCG under conditions permitting formation of a complex between the antibody and the molecular isoform of hCG; and (b) measuring the amount of complexes formed, thereby determining the amount of the molecular isoform of hCG in the sample.

6. A method according to claim 5 further comprising (c) comparing the amount of molecular isoform of hCG in the sample determined in step (b) with either (i) the amount determined for temporally matched, normal samples or (ii) the amount determined for samples obtained from non-pregnant subject(s), wherein the relative absence of the molecular isoform of hCG in the sample indicates that the embryo is relatively viable.

7. A method according to claim 5 further comprising (c) comparing the amount of molecular isoform of hCG in the sample determined in step (b) with either (i) the amount determined for temporally matched, normal samples or (ii) the amount determined for samples obtained from non-pregnant subject(s), wherein the relative abundance of the molecular isoform of hCG in the sample indicates that the embryo is relatively viable.

8. A method according to claim 1 wherein the embryo is between about 3 and about 5 days old.

9. A method of predicting the likelihood of a positive pregnancy outcome in a female subject comprising determining the presence of one or more pregnancy associated markers in a sample.

10. A method according to claim 9 wherein the pregnancy associated marker is a molecular isoform of hCG.

11. A method according to claim 9 wherein the sample is selected from the group consisting of an in vitro growth media, amniotic fluid, plasma, serum, urine and blood.

12. A method according to claim 9 wherein determining the presence of one or more pregnancy associated markers in a sample is performed by Matrix assisted laser desorption ionization (MALDI) mass spectrometry.

13. A method according to claim 9 wherein determining the presence of one or more pregnancy associated markers in a sample is performed by contacting the sample with an antibody which specifically binds to a molecular isoform of hCG under conditions permitting formation of a complex between the antibody and the molecular isoform of hCG; (b) measuring the amount of complexes formed, thereby determining the amount of the molecular isoform of hCG in the sample.

14. A method according to claim 13 further comprising (c) comparing the amount of molecular isoform of hCG in the sample determined in step (b) with either (i) the amount determined for temporally matched, normal pregnant samples or (ii) the amount determined for samples obtained from non-pregnant subject(s), wherein the relative abundance of the molecular isoform of hCG in the sample indicates a greater than normal likelihood of a positive pregnancy.

15. A method of predicting the likelihood of a negative pregnancy outcome in a female subject comprising determining the presence of one or more pregnancy associated markers in a sample.

16. A method according to claim 15 wherein the pregnancy associated marker is a molecular isoform of hCG.

17. A method according to claim 15 wherein the sample is selected from the group consisting of an in vitro growth media, amniotic fluid, plasma, serum, urine and blood.

18. A method according to claim 15 wherein determining the presence of one or more pregnancy associated markers in a sample is performed by Matrix assisted laser desorption ionization (MALDI) mass spectrometry.

19. A method according to claim 15 wherein determining the presence of one or more pregnancy associated markers in a sample is performed by contacting the sample with an antibody which specifically binds to the molecular isoform of hCG under conditions permitting formation of a complex between the antibody and the molecular isoform of hCG; (b) measuring the amount of complexes formed, thereby determining the amount of the molecular isoform of hCG in the sample.

20. A method according to claim 13 further comprising (c) comparing the amount of molecular isoform of hCG in the sample determined in step (b) with either (i) the amount determined for temporally matched, normal pregnant samples or (ii) the amount determined for samples obtained from non-pregnant subject(s), wherein the relative absence of the molecular isoform of hCG in the sample indicates a greater than normal likelihood of a negative pregnancy.

21. A diagnostic kit for predicting viability of an embryo or for determining the likelihood of a positive outcome for a pregnancy comprising a means for detecting the presence or the quantity of a pregnancy associated marker and instructions correlating the presence or the quantity of the pregnancy associated marker with the likelihood that an embryo will become viable or that a pregnancy will result in a positive outcome.
